(19) United States
(12) Patent Application Publication
(10) Pub. No.: US 2019/0248958 A1
(43) Pub. Date: Aug. 15, 2019

(54) PREPARATION METHOD OF OLIGOMER ADDITIVE, OLIGOMER ADDITIVE, AND LITHIUM BATTERY

(71) Applicant: National Taiwan University of Science and Technology, Taipei (TW)

(72) Inventors: Fu-Ming Wang, Taipei (TW); Chorng-Shyan Chern, Taipei (TW)

(73) Assignee: National Taiwan University of Science and Technology, Taipei (TW)

(21) Appl. No.: 15/924,288

(22) Filed: Mar. 19, 2018

(30) Foreign Application Priority Data
Feb. 13, 2018 (TW) 107105219

(51) Int. Cl. C08G 73/10; C07D 207/40; H01M 10/0567; H01M 10/0568; H01M 10/052; H01M 4/62

(52) U.S. Cl. CPC C08G 73/1003; C07D 207/40; H01M 10/0567; H01M 2300/0025; H01M 10/052; H01M 4/62; H01M 10/0568

(57) ABSTRACT

A preparation method of an oligomer additive including the following steps is provided. A compound (A) having a secondary amine and a basic compound (B) are reacted. Next, a product of the reaction and a compound (C) having an unsaturated carbon-carbon double bond are reacted in a solvent. When the oligomer additive prepared by the method is applied in the cathode of a lithium battery, the cathode electrode core structure can be effectively protected from an environment condition such as high temperature, droppage, or deformation by external force. Moreover, the lithium battery cycle life can be maintained.

### CROSS-REFERENCE TO RELATED APPLICATION

This application claims the priority benefit of Taiwan application serial no. 107105219, filed on Feb. 13, 2018. The entirety of the above-mentioned patent application is hereby incorporated by reference herein and made a part of this specification.

### BACKGROUND OF THE INVENTION

### Field of the Invention

The invention relates to a preparation method of an oligomer additive, and more particularly, to a preparation method of an oligomer additive for a lithium battery.

### Description of Related Art

Since primary batteries are not environment-friendly, the market demand for secondary lithium batteries with characteristics such as rechargeability, light weight, high voltage value, and high energy density has been growing in recent years. The secondary lithium battery is a battery for which cyclic charging and discharging can occur in the cathode and anode materials. The current performance requirements for secondary lithium batteries such as light weight, durability, high voltage, high energy density, and high safety have become higher. In particular, secondary lithium batteries have very high potential in the application and expandability in light electric vehicles, electric vehicles, and the large power storage industry.

However, lithium transition metal oxide is still largely used as the cathode material in commercialized secondary lithium batteries on the general market, and the main drawback thereof is that, in high-temperature applications, the lithium salt in the battery is readily pyrolyzed, and therefore the structure of the cathode material is damaged. As a result, oxygen in the lithium metal oxide structure is readily released to participate in a combustion reaction, which is one of the main reasons causing the explosion, swelling, and performance degradation of secondary lithium batteries. Therefore, continuously maintaining the structural stability of the lithium salt in high-temperature applications is one of the desired goals of those skilled in the art.

Currently, most proposed solutions include, for instance, switching to other cathode materials with better stability, adding different types of additives in the electrolyte solution to improve the surface properties of the cathode material, or adding a cooling mechanism in the battery module. However, these methods all complicate the battery preparation steps.

To solve the technical issues above, in the invention, an oligomer additive is prepared by a specific method, and the oligomer additive can be directly added in a slurry of an existing commercialized cathode lithium transition metal oxide to significantly increase the performance thereof, thus resulting in very high applicability.

### SUMMARY OF THE INVENTION

The invention provides a preparation method of an oligomer additive. When the oligomer additive prepared by the method is applied in the cathode of a lithium battery, the cathode electrode core structure can be effectively protected from an environment condition such as high temperature, droppage, or deformation by external force. Moreover, the lithium battery cycle life can be maintained.

The preparation method of the oligomer additive of the invention includes the following steps. A compound (A) having a secondary amine and a basic compound (B) are reacted. Next, the resulting product and a compound (C) having an unsaturated carbon-carbon double bond are reacted in a solvent.

In an embodiment of the invention, the mass ratio of the compound (A) having the secondary amine and the basic compound (B) is between 1:5 and 1:20.

In an embodiment of the invention, based on a total weight of 100 parts by weight of the oligomer additive, an amount of the compound (A) having the secondary amine is 0.5 parts by weight to 5 parts by weight, an amount of the basic compound (B) is 5 parts by weight to 50 parts by weight, and an amount of the compound (C) having the unsaturated carbon-carbon double bond is 2 parts by weight to 20 parts by weight.

In an embodiment of the invention, the compound (A) having the secondary amine has three or more than three secondary amines.

In an embodiment of the invention, the compound (A) having the secondary amine is cyanuric acid (CA).

In an embodiment of the invention, the basic compound (B) is dimethyl sulfoxide (DMSO).

In an embodiment of the invention, the compound (C) having the unsaturated carbon-carbon double bond is monomaleimide or bismaleimide, and the monomaleimide is at least one selected from the group consisting of N-phenylmaleimide, N-(o-methylphenyl)-maleimide, N-(m-methylphenyl)-maleimide, N-(p-methylphenyl)-maleimide, N-cyclohexylmaleimide, maleimidophenol, maleimidobenzocyclobutene, phosphorus-containing maleimide, phosphonate-containing maleimide, siloxane-containing maleimide, N-(4-tetrahydropyranyl-oxyphenyl)maleimide, and 2,6-xylylmaleimide, and the bismaleimide has a structure represented by formula 1:

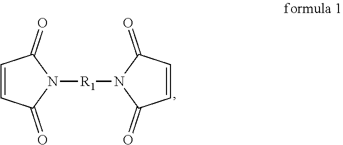

wherein R₁ is:

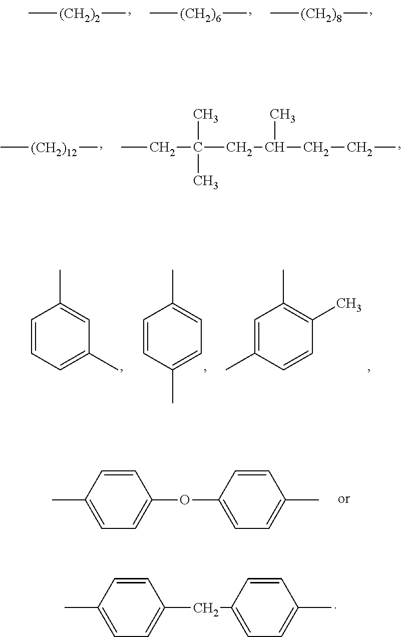

The oligomer additive of the invention is prepared by the preparation method of the oligomer additive, wherein after the compound (A) having the secondary amine and the basic compound (B) are reacted, a reaction with the compound (C) having the unsaturated carbon-carbon double bond is performed in a solvent, and the mass ratio of the compound (A) having the secondary amine and the basic compound (B) is between 1:5 and 1:20.

A lithium battery of the invention includes an anode, a cathode, a separator, an electrolyte solution, and a package structure. The cathode and the anode are separately disposed, and the cathode includes the oligomer additive above. The separator is disposed between the anode and the cathode, and the separator, the anode, and the cathode define a housing region. The electrolyte solution is disposed in the housing region. The package structure covers the anode, the cathode, and the electrolyte solution.

In an embodiment of the invention, the electrolyte solution includes an organic solvent, lithium salt, and an additive.

In an embodiment of the invention, the additive includes monomaleimide, polymaleimide, bismaleimide, polybismaleimide, a copolymer of bismaleimide and monomaleimide, vinylene carbonate, or a mixture thereof.

Based on the above, the oligomer additive of the invention is prepared by first reacting the compound (A) having the secondary amine and the basic compound (B) and then performing a reaction with the compound (C) having the unsaturated carbon-carbon double bond in a solvent. The battery properties are not affected, and instead the drawbacks in prior art above can be overcome to increase the overall battery energy density. Moreover, by adjusting the battery performance, the battery cycle life can be improved.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a cross section of a lithium battery according to an embodiment of the invention.

FIG. 2 shows the relationship between number of charge and discharge cycles and discharge capacity of the lithium batteries of experimental example 1, comparative example 1, and comparative example 2 at room temperature.

FIG. 3 shows the charge and discharge curves of the lithium batteries of example 1 and comparative example 3 at room temperature.

FIG. 4 is an AC impedance spectrum of the lithium batteries of example 1 and comparative example 3.

### DESCRIPTION OF THE EMBODIMENTS

In the present specification, a range represented by “a numerical value to another numerical value” is a schematic representation for avoiding listing all of the numerical values in the range in the specification. Therefore, the recitation of a specific numerical range covers any numerical value in the numerical range and a smaller numerical range defined by any numerical value in the numerical range, as is the case with the any numerical value and the smaller numerical range stated explicitly in the specification.

To prepare an oligomer additive that can effectively protect the cathode electrode core structure of the lithium battery and maintain battery cycle life in any environment, the invention provides a preparation method of the oligomer additive that can achieve the advantages above. In the following, embodiments are provided as examples of actual implementation of the invention.

<Preparation Method of Oligomer Additive>

An embodiment of the invention provides a preparation method of the oligomer additive including the following steps. A compound (A) having a secondary amine and a basic compound (B) are reacted. Next, the resulting product and a compound (C) having an unsaturated carbon-carbon double bond are reacted in a solvent.

<Compound (A) having a Secondary Amine>

In the present embodiment, the quantity of the secondary amine and the type of the compound are not limited as long as the compound (A) has the secondary amine. For instance, the compound (A) can be cyanuric acid; barbituric acid; aliphatic secondary amine such as dibutylamine, diamylamine, dihexylamine, di-(2-ethylhexyl)amine, diheptylamine, dioctylamine, dinonylamine, didecylamine, henicosylamine, behenylamine, tricosylamine, tetradecylamine, pentacosylamine, diisobutylamine, diisoamylamine, diisohexylamine, diisoheptylamine, diisooctylamine, diisononylamine, diisodecylamine, diisoundecylamine, diisododecylamine, diisotridecylamine, diisotetradecylamine, diisopentadecylamine, butylpentylamine, butylhexylamine, hexylpentylamine, butyloctylamine, or nonyloctylamine; aromatic secondary amine such as dibenzylamine, bis-(methylbenzyl)amine, bis-(methoxybenzyl)amine, di-(ethylbenzyl)amine, di-(ethoxybenzyl)amine, di-(butylbenzyl)amine, bis-(butoxybenzyl)amine, phenethylamine, bis-(methylphenethyl)amine, bis-(methoxyphenethyl)amine, bis-(ethylphenethyl)amine, di-(ethoxyphenethyl)amine, bis-(butylphenethyl)amine, bis-(butoxyphenethyl)amine, diallyl amine, bis-(methylbenzeneallyl)amine, bis-(methoxybenzyl)amine, di-(ethylbenzene allyl)amine, di-(ethoxyphenylallyl)amine, bis-(butylbenzyl)amine, or bis-(butoxybenzyl)amine, or a combination thereof. From the standpoint of increasing reaction rate, the compound (A) having the secondary amine preferably has three or three or more secondary amines, and in another embodiment, the compound (A) having the secondary amine is preferably cyanuric acid.

<Basic Compound (B)>

In the present embodiment, the type of the compound (B) is not limited as long as the compound (B) is basic. For instance, the compound (B) can be dimethyl sulfoxide (DMSO), dimethyl formamide (DMF), N,N-dimethylethyl amine (DMAc), N-methylpyrrolidone (NMP), γ-butyrolactone (GBL), other basic compounds, or a combination thereof. From the standpoint of increasing reaction rate, the basic compound (B) is preferably DMSO.

Moreover, in the present embodiment, the method of preparing the oligomer additive includes first reacting the compound (A) having the secondary amine and the basic compound (B), wherein the proportion of the compound (A) having the secondary amine is not limited as long as the compound (A) having the secondary amine can be dissolved in the basic compound (B). For instance, the mass ratio of the compound (A) having the secondary amine and the basic compound (B) is preferably between 1:5 and 1:20, more preferably between 1:7 and 1:7.5. Via the proportions above, better solubility can be achieved to increase reaction rate and lower cost.

Specifically, in the present embodiment, the cyanuric acid having three secondary amines has three sets of ═N-groups and OH groups, which is not productive to the reaction with the maleimide used as the compound (C) having the unsaturated carbon-carbon double bond, with the reason being the low activation energy of polymer reactions, and therefore thermal stability is poor. Therefore, in the invention, cyanuric acid and DMSO used as the basic compound are reacted first before the cyanuric acid and the maleimide imine are reacted to react the three ═N-groups in the cyanuric acid into —NH-groups and react the OH group into a ═O group. Via the reaction above, the cyanuric acid and the unsaturated carbon-carbon double bond compound can be reacted at a higher temperature to increase the overall polymer reaction efficiency and thermal stability.

<Compound (C) having an Unsaturated Carbon-Carbon Double Bond>

In the present embodiment, the type of the compound (C) is not limited as long as the compound (C) has the unsaturated carbon-carbon double bond. For instance, the compound (C) can be an unsaturated carboxylic acid such as acrylic acid, methacrylic acid, vinyl acetate, 2-pentenoic acid, 3-pentenoic acid, 5-hexenoic acid, 9-decenoic acid, or 9-undecenoic acid; acyl chloride or chloroformate such as propylene chloride, methacryloyl chloride, sorbitol acyl chloride, allyl alcohol chloroformate, isopropenyl phenol chloroformate, or hydroxystyrene chloroformate; phenol having an unsaturated acid or maleic acid, fumaric acid, or maleic anhydride such as isopropenyl phenol, hydroxystyrene, hydroxyphenyl maleimide, maleimide imine, allyl hydroxybenzoate, or methyl allyl hydroxybenzoate. From the standpoint of increasing reaction rate, the compound (C) having the unsaturated carbon-carbon double bond is preferably maleimide imine.

In the present embodiment, the maleimide is, for instance, monomaleimide or bismaleimide. The monomaleimide is, for instance, selected from the group consisting of N-phenylmaleimide, N-(o-methylphenyl)-maleimide, N-(m-methylphenyl)-maleimide, N-(p-methylphenyl)-maleimide, N-cyclohexylmaleimide, maleimidophenol, maleimidobenzocyclobutene, phosphorus-containing maleimide, phosphonate-containing maleimide, siloxane-containing maleimide, N-(4-tetrahydropyranyl-oxyphenyl)maleimide, and 2,6-xylylmaleimide. The bismaleimide can have the structure represented by formula 1:

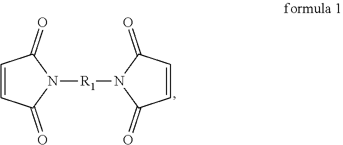

wherein R₁ is:

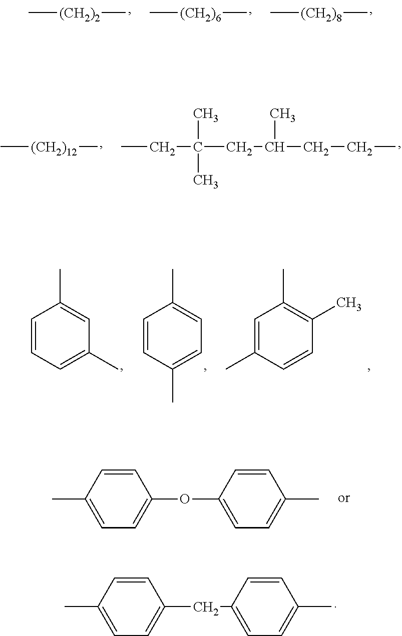

In the present embodiment, a Michael addition reaction occurs between the maleimide and the cyanuric acid.

In the present embodiment, the proportions of each component in the preparation method are not particularly limited as long as the resulting oligomer additive product has the technical effects of the present application above. For instance, based on a total weight of 100 parts by weight of the oligomer additive, the amount of the compound (A) having the secondary amine is preferably 0.5 parts by weight to 5 parts by weight, more preferably 1 part by weight to 3 parts by weight; the amount of the basic compound (B) is preferably 5 parts by weight to 50 parts by weight, more preferably 10 parts by weight to 15 parts by weight; and the amount of the compound (C) having the unsaturated carbon-carbon double bond is preferably 2 parts by weight to 20 parts by weight, more preferably 5 parts by weight to 10 parts by weight. The oligomer additive prepared at the proportions above can increase the overall battery energy density so as to improve the battery cycle life.

<Solvent>

In the invention, the solvent can be an organic solvent, and examples thereof include N-methyl pyrollidone (NMP), γ-butylrolactone (GBL), and propylene carbonate (PC). The solvents can be used alone or in combination. In another embodiment, the solvent is preferably NMP alone.

The solvent of the invention is preferably a different compound than the basic compound (B), but the same compound as the basic compound (B) can also be used.

<Oligomer Additive>

In the present embodiment, in the oligomer additive prepared by the preparation method of the oligomer additive, the mass ratio of the compound (A) having the secondary amine and the basic compound (B) is preferably between 1:5 and 1:20, more preferably between 1:7 and 1:7.5.

It should be mentioned that, the oligomer additive can be applied in the cathode material of a lithium battery. More specifically, since the oligomer additive has good thermal reactivity, a protective layer is formed on the cathode material surface. The protective layer can effectively prevent damage to the cathode structure from a high-temperature environment for the following reasons. As described above, the oligomer additive has a highly-branched structure, and therefore a stable organic polymer spread on the surface thereof can be formed with the metal oxide in a regular cathode material. Moreover, since the oligomer additive has high thermal reactivity, high thermal stability, and a rigid chemical structure, the resulting protective layer can have high thermal stability. As a result, the lithium battery having a cathode material including the oligomer additive can still have good capacitance, battery efficiency, and safety in a high-temperature environment, and the battery cycle life can be improved.

Moreover, as described above, by first reacting the basic compound (B) and the compound (A) having the secondary amine and then reacting the product thereof with the compound (C) having the unsaturated carbon-carbon double bond in a solvent, the reaction rate, conversion rate, and structure . . . etc. of the oligomer additive can be regulated to increase the overall battery energy density and improve battery cycle life as a result.

<Lithium Battery>

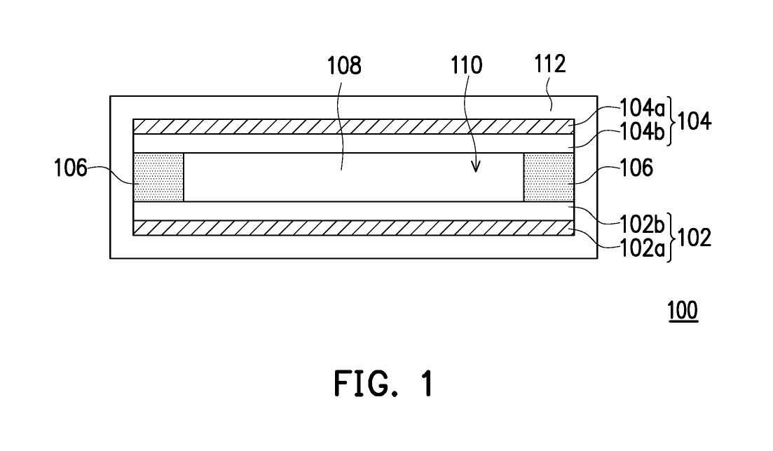

Another embodiment of the invention provides a lithium battery including the oligomer additive in any one of the above embodiments. In the following, description is provided with reference to FIG. 1.

FIG. 1 is a cross section of a lithium battery according to an embodiment of the invention.

Referring to FIG. 1, a lithium battery 100 includes an anode 102, a cathode 104, a separator 106, an electrolyte solution 108, and a package structure 112.

In the present embodiment, the anode 102 includes an anode metal foil 102a and an anode material 102b, wherein the anode material 102b is disposed on the anode metal foil 102a through coating or sputtering. The anode metal foil 102a is, for instance, a copper foil, an aluminum foil, a nickel foil, or a high-conductivity stainless steel foil. The anode material 102b is, for instance, carbide or metal lithium. The carbide used as the anode material 102b is, for instance, carbon powder, graphite, carbon fiber, carbon nanotube, graphene, or a mixture thereof. However, in other embodiments, the anode 102 can also only include the anode material 102b.

The cathode 104 and the anode 102 are separately disposed. The cathode 104 includes a cathode metal foil 104a and a cathode material 104b, wherein the cathode material 104b is disposed on the cathode metal foil 104a through coating. The cathode metal foil 104a is, for instance, a copper foil, an aluminum foil, a nickel foil, or a high-conductivity stainless steel foil. The cathode material 104b includes the oligomer additive in any of the embodiments above and lithium mixed transition metal oxide, wherein based on a total weight of 100 parts by weight of the cathode material 104b, the content of the oligomer additive is 0.5 parts by weight to 5 parts by weight, preferably 1 part by weight to 3 parts by weight, and the content of the lithium mixed transition metal oxide is, for instance, 80 parts by weight to 95 parts by weight. If the content of the oligomer additive is less than 0.5 parts by weight, then the battery safety characteristic is not significant; and if the content of the oligomer additive is higher than 5 parts by weight, then battery cycle life is poor. The oxide of lithium mixed with a transition metal is, for instance, LiMnO₂, LiMn₂O₄, LiCoO₂, Li₂Cr₂O₇, Li₂CrO₄, LiNiO₂, LiFeO₂, LiFePO₄, LiMn_{0.5}Ni_{0.5}O₂, LiMn_{1/3}Co_{1/3}Ni_{1/3}O₂, LiMc_{0.5}Mn_{1.5}O₄, or a combination thereof, wherein 0<x<1, and Mc is a divalent metal.

Moreover, in an embodiment, the lithium battery 100 can further include a polymer binder, and the polymer binder reacts with the anode 102 and/or the cathode 104 to increase the mechanical properties of the electrode(s). Specifically, the anode material 102b can be adhered to the anode metal foil 102a through the polymer binder, and the cathode material 104b can be adhered to the cathode metal foil 104a through the polymer binder. The polymer binder is, for instance, polyvinylidene difluoride (PVDF), styrene-butadiene rubber (SBR), polyamide, melamine resin, or a combination thereof.

The separator 106 is disposed between the anode 102 and the cathode 104, and the separator 106, the anode 102, and the cathode 104 define a housing region 110. The material of the isolation film 106 is, for instance, an insulating material, and the insulating material can be polyethylene (PE), polypropylene (PP), or a multilayer composite structure of the materials, such as PE/PP/PE.

In the present embodiment, the electrolyte solution 108 is disposed in the housing region 110, and the electrolyte solution 108 includes an organic solvent, lithium salt, and other additives. In particular, the content of the organic solvent in the electrolyte solution 108 is 55 wt % to 90 wt %, the content of the lithium salt in the electrolyte solution 108 is 10 wt % to 35 wt %, and the content of the other additives in the electrolyte solution 108 is 0.05 wt % to 10 wt %. However, in other embodiments, the electrolyte solution 108 may also not include other additives.

The organic solvent is, for instance, γ-butyl lactone, ethylene carbonate (EC), propylene carbonate, diethyl carbonate (DEC), propyl acetate (PA), dimethyl carbonate (DMC), ethylmethyl carbonate (EMC), or a combination thereof.

The lithium salt is, for instance, LiPF₆, LiBF₄, LiAsF₆, LiSbF₆, LiClO₄, LiAlCl₄, LiGaCl₄, LiNO₃, LiC(SO₂CF₃)₃, LiN(SO₂CF₃)₂, LiSCN, LiO₃SCF₂CF₃, LiC₆F₅SO₃, LiO₂CCF₃, LiSO₃F, LiB(C₆H₅)₄, LiCF₃SO₃, or a combination thereof.

The other additives include, for instance, monomaleimide, polymaleimide, bismaleimide, polybismaleimide, a copolymer of bismaleimide and monomaleimide, vinylene carbonate (VC), or a mixture thereof. The monomaleimide is, for instance, selected from the group consisting of N-phenylmaleimide, N-(o-methylphenyl)-maleimide, N-(m-methylphenyl)-maleimide, N-(p-methylphenyl)-maleimide, N-cyclohexylmaleimide, maleimidophenol, maleimidobenzocyclobutene, phosphorus-containing maleimide, phosphonate-containing maleimide, siloxane-containing maleimide, N-(4-tetrahydropyranyl-oxyphenyl)maleimide, and 2,6-xylylmaleimide. The bismaleimide can have the structure represented by formula 1 above.

The package structure 112 is used to cover the anode 102, the cathode 104, and the electrolyte solution 108. The material of the package structure 112 is, for instance, aluminum foil.

It should be mentioned that, by directly adding the oligomer additive of any of the embodiments above in the slurry of the cathode material 104b of the lithium battery 100 for mixing, the oligomer additive can be effectively spread on the particle surface of the cathode material 104b. A protective layer is formed by the cover of the oligomer additive, and the battery can still be effectively charged and discharged. As a result, the oligomer additive not only does not affect battery properties in any environment, but instead effectively protects the cathode material 104b of the lithium battery 100 and increases the overall battery energy density, such that battery cycle life can be maintained, and the performance of the lithium battery 100 can be significantly increased as a result, thus resulting in very high applicability.

Moreover, the cathode 104 having a protective layer in the lithium battery 100 can be formed by directly adding the oligomer additive in the cathode material in a current battery manufacturing process. Therefore, the battery cycle life of the lithium battery 100 can be effectively maintained in any environment without modifying any battery design, electrode material, and electrolyte solution.

In the following, the effects of the oligomer additive of the invention are described with experimental examples and comparative examples.

### EXAMPLE 1

First, 1.4827 g of cyanuric acid was added in 11.25 g of DMSO. After stirring for 5 minutes, the cyanuric acid was completely dissolved at room temperature, and the product thereof was designated DMSO/CA. Next, all of the DMSO/CA, 6.4121 g of maleimide imine (molar ratio: 2:3), and 150 g of NMP (solvent) were added in a reactor, and the mixture was stirred and reacted at a temperature of 130° C. for 1 hour and then placed in an ice bath to obtain an oligomer additive. The mixture was then added in a cathode slurry at a proportion of 1.5 parts by weight.

### COMPARATIVE EXAMPLE 1

1.4827 g of cyanuric acid, 6.4121 g of maleimide imine (molar ratio: 2:3), and 150 g of NMP (solvent) were directly added in a reactor, and the components were stirred and reacted at a temperature of 130° C. for 1 hour and then placed in an ice bath to obtain an oligomer additive. The mixture was then added in a cathode slurry at a proportion of 1.5 parts by weight.

### COMPARATIVE EXAMPLE 2

The lithium battery does not contain any additive.

### COMPARATIVE EXAMPLE 3

First, 1.4827 g of barbituric acid (BTA) was added in 11.25 g of DMSO until all of the BTA was dissolved. The product thereof was designated DMSO/BTA. Next, the DMSO/BTA and maleimide imine were added in a reactor at a proportion of 1:2 molar ratio, and the components were reacted at a temperature of 130° C. for 1 hour, and then added in a cathode slurry at a proportion of 1.5 parts by weight.

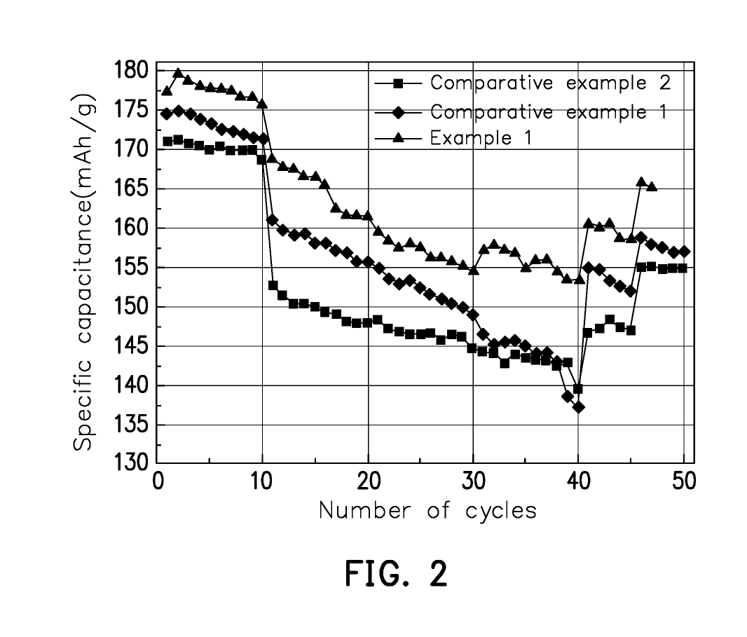

The oligomer additives of example 1 and comparative examples 1 to 2 were respectively applied in the cathode material of the same lithium battery, and a cycle life test was performed on the lithium battery. FIG. 2 shows the relationship between the number of charge and discharge cycles and discharge capacity of the lithium batteries having the oligomer additive of example 1 and comparative example 1 and a lithium battery without the oligomer additive (comparative example 2) at room temperature. It is clear from FIG. 2 that, when the lithium battery has the oligomer additive of the invention (example 1), in comparison to the lithium battery without the oligomer additive (comparative example 2), the battery discharge capacity thereof can still be maintained at a higher battery discharge capacity after 10 cycles (about 172 mAh/g to 176 mAh/g). Moreover, after repeated high-rate charge and discharge, when the lithium battery has the oligomer additive of the invention (example 1), in comparison to the lithium battery without the oligomer additive (comparative example 2), the battery discharge capacity thereof can still be maintained at a higher battery discharge capacity after 50 cycles (about 165 mAh/g to 157 mAh/g), indicating the oligomer additive of the invention not only does not affect battery properties, but can overcome the drawbacks of the prior art and slightly increase overall battery energy density.

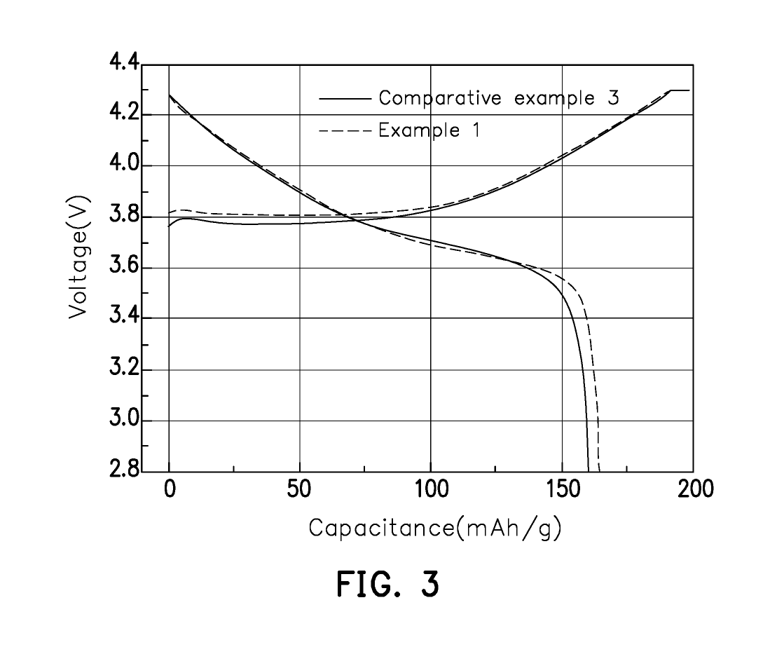

Next, a charge and discharge performance test was performed on the lithium batteries of example 1 and comparative example 3, and the measurement results thereof are shown in FIG. 3.

Charge and discharge was performed on the lithium batteries of example 1 and comparative example 3 at fixed current/voltage at room temperature (30° C.) using a potentiostat (made by Biologic Corporation, model: VMP3). First, the batteries were charged to 4.3 V with a constant current of 0.2 C until the current was less than or equal to 0.01 C. Then, the batteries were discharged to the cut-off voltage (3 V) with a constant current of 0.2 C. FIG. 3 shows the charge and discharge curves of the lithium batteries of example 1 and comparative example 3 at room temperature.

It can be known from FIG. 3 that, the 160.3 mAh/g discharge capacity of the lithium battery of comparative example 3 is slightly less than the 164.5 mAh/g discharge capacity of the lithium battery of example 1. In other words, in comparison to the lithium battery of comparative example 3, at room temperature, the lithium battery having the oligomer additive of the cathode of example 1 has higher discharge capacity. In other words, example 1 having three or more than three secondary amine compounds has higher discharge capacity compared to comparative example 3 having two secondary amine compounds.

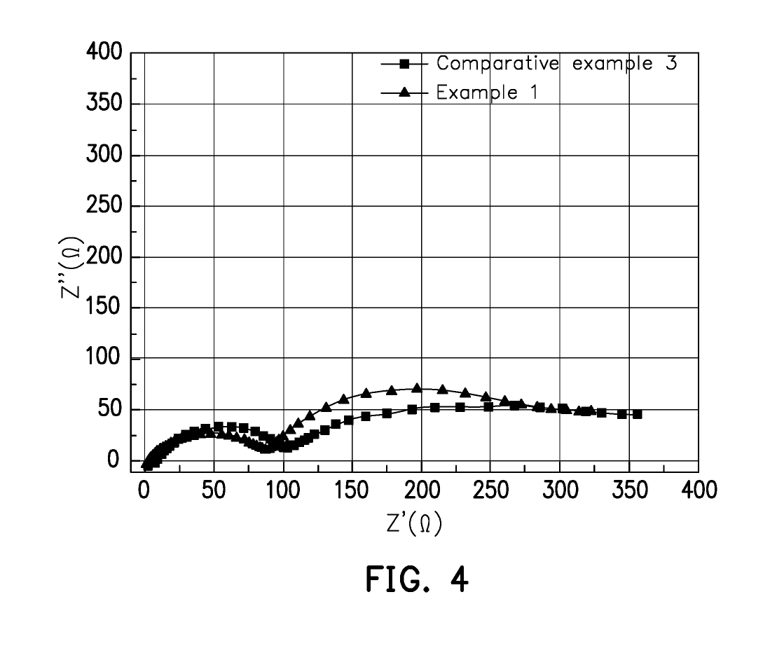

Moreover, the AC impedance spectrums of the lithium batteries of experimental example 1 and comparative example 3 were recorded via electrochemical impedance spectrometry (EIS), and the results are shown in FIG. 4. It can be observed that the AC impedance performances of the lithium batteries of experimental example 1 and comparative example 3 both respectively show 2 semicircular curves. The curves represent the effects of the lithium batteries and different electron transfer mechanism results, wherein the data of experimental example 1 has a relatively low resistance compared to that of comparative example 3. Therefore, the lithium battery of experimental example 1 has a relatively low body resistance. In other words, example 1 having three or more than three secondary amine compounds has a relatively low resistance compared to comparative example 3 having two secondary amine compounds.

### EXAMPLE 2

2 g of cyanuric acid was respectively added in 14 g and 15 g of DMSO, and after stirring for 5 minutes, the mixture was placed at room temperature for solubility testing. The results are shown in Table 1 below:

TABLE 1

| Proportion | Dissolution |
|---|---|
| 2 g of cyanuric acid/14 g of | Soluble (4 hours to dissolution, close to |
| DMSO | saturation concentration) |
| 2 g of cyanuric acid/15 g of | Soluble (1 hour to dissolution) |
| DMSO | |

It can be known from Table 1 that, when 2 g of cyanuric acid and 14 g of DMSO are reacted, the saturation concentration is close, and dissolution can be achieved after 4 hours. When 2 g of cyanuric acid and 15 g of DMSO are reacted, dissolution can be achieved at 1 hour. In other words, cyanuric acid can be dissolved in DMSO under all of the proportions of cyanuric acid and DMSO above, which is applicable to the invention of the present application.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A preparation method of an oligomer additive, comprising the steps of reacting a compound (A) having a secondary amine and a basic compound (B) are reacted. Next, the resulting product and a compound (C) having an unsaturated carbon-carbon double bond are reacted in a solvent.

2. The preparation method of the oligomer additive of claim 1, wherein a mass ratio of the compound (A) having the secondary amine and the basic compound (B) is between 1:5 and 1:20.

3. The preparation method of the oligomer additive of claim 1, wherein based on a total weight of 100 parts by weight of the oligomer additive, an amount of the compound (A) having the secondary amine is 0.5 parts by weight to 5 parts by weight, an amount of the basic compound (B) is 5 parts by weight to 50 parts by weight, and an amount of the compound (C) having the unsaturated carbon-carbon double bond is 2 parts by weight to 20 parts by weight.

4. The preparation method of the oligomer additive of claim 1, wherein the compound (A) having the secondary amine has three or more than three secondary amines.

5. The preparation method of the oligomer additive of claim 1, wherein the basic compound (B) is dimethyl sulfoxide.

6. The preparation method of the oligomer additive of claim 1, wherein the compound (C) having the unsaturated carbon-carbon double bond is monomaleimide or bismaleimide, and the monomaleimide is at least one selected from the group consisting of N-phenylmaleimide, N-(o-methylphenyl)-maleimide, N-(m-methylphenyl)-maleimide, N-(p-methylphenyl)-maleimide, N-cyclohexylmaleimide, maleimidophenol, maleimidobenzocyclobutene, phosphorus-containing maleimide, phosphonate-containing maleimide, siloxane-containing maleimide, N-(4-tetrahydropyranyl-oxyphenyl)maleimide, and 2,6-xylylmaleimide, and the bismaleimide has a structure represented by formula 1: 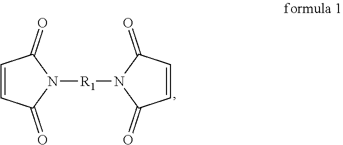
wherein R₁ is

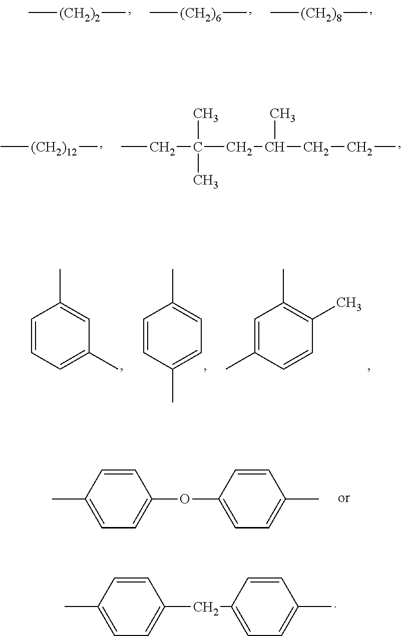

7. An oligomer additive prepared by the preparation method of the oligomer additive of claim 1, wherein a compound (A) having a secondary amine and a basic compound (B) are reacted, and then a reaction with a compound (C) having an unsaturated carbon-carbon double bond is performed in a solvent;
a mass ratio of the compound (A) having the secondary amine and the basic compound (B) is between 1:5 and 1:20.

8. A lithium battery, comprising:
an anode;
a cathode disposed separately from the anode, wherein the cathode comprises the oligomer additive of claim 7;
a separator disposed between the anode and the cathode, wherein the separator, the anode, and the cathode define a housing region;
an electrolyte solution disposed in the housing region; and
a package structure covering the anode, the cathode, and the electrolyte solution.

9. The lithium battery of claim 8, wherein the electrolyte solution comprises an organic solvent, lithium salt, and an additive.

10. The lithium battery of claim 8, wherein the additive comprises monomaleimide, polymaleimide, bismaleimide, polybismaleimide, a copolymer of bismaleimide and monomaleimide, vinylene carbonate, or a mixture thereof.
